(19) United States
(12) Patent Application Publication
(10) Pub. No.: US 2001/0029029 A1
(43) Pub. Date: Oct. 11, 2001

(54) Cellular analysis instrument

(76) Inventors: Peter P. Gombrich, Chicago, IL (US); Richard A. Domanik, Libertyville, IL (US); George Gorodeski, Beechwood, OH (US)

Correspondence Address: MERCHANT & GOULD PC, P.O. BOX 2903, MINNEAPOLIS, MN, 55402-0903, US

(21) Appl. No.: 09/725,795

(22) Filed: Nov. 29, 2000

Related U.S. Application Data
(60) Provisional application No. 60/172,401, filed on Dec. 17, 1999.
(60) Provisional application No. 60/183,140, filed on Feb. 17, 2000.

(51) Int. Cl. G01N 33/48; C12M 1/34

(52) U.S. Cl. 435/40.5; 435/287.1; 356/300

(57) ABSTRACT

Cellular samples can be evaluated in a manner in which the spatial relationships existing between said cells in-vivo are preserved and can be used to guide subsequent follow-up and treatment where abnormalities are detected. An instrument for analyzing cervical cells includes receiving means for receiving a topological array of cervical cells, reagent application means for applying one or more reagents to the topological array of cervical cells, analysis means for optically analyzing the topological array of cervical cells, and reporting means for reporting results of the analysis means.

### RELATED APPLICATIONS

This application claims priority to provisional application Ser. No. 60/172,401, filed Dec. 17, 1999, entitled “INSTRUMENT FOR ANALYZING TISSUE COLLECTED BY IN-VIVO SAMPLE COLLECTOR”, and provisional application Ser. No. 60/183,140, filed Feb. 17, 2000, entitled “CELL RECOVERY DEVICE”, which applications are specifically incorporated by reference herein.

### TECHNICAL FIELD

The invention relates generally to cervical cell sampling and analysis and more specifically to devices and methods whereby cervical cell samples can be accurately attained and rapidly analyzed.

### BACKGROUND

Cervical cancer is a leading form of cancer among women. In the United States alone, there are believed to be more than two million cases of precancerous cervical abnormalities annually. The U.S. also sees, on average, about sixty five thousand cases of cervical carcinoma and about sixteen thousand cases of invasive cervical cancer. Although screening is less common outside the Unites States, nearly half a million cases of cervical cancer are detected each year around the world.

Cervical cancer frequently begins as a precancerous lesion of the cervix. These lesions are also known as cervical intraepithelial neoplasia. If left untreated, these lesions can deepen over time and ultimately develop into an invasive cancer of the cervix and associated tissues. Fortunately, early detection followed by appropriate treatment results in a very high cure rate for cervical cancer.

Therefore, it is quite important that at least certain factions of the female population undergo regular screening. These factions include patients with previous cervical abnormalities and those who have a family history of cervical abnormalities. Women who are sexually active are at greater risk and should undergo regular screening, as are those who test positive for HPV (human papillomavirus). This is a sexually transmitted virus that in some forms can cause genital warts.

During the 1940's, Dr. George Papanicolaou developed a screening test which bears his name and which has become the most widely used screening technique for detecting abnormal cervical cells. Today, this test is known more commonly as the PAP test or the PAP smear test. Typically, the PAP test is performed in the physician's office as part of a woman's routine gynecological examination. The test involves collecting cervical cells via a brush, stick or swab that is used to loosen and then collect cells that can be examined microscopically.

Cervical samples taken for the purposes of Pap testing are deposited on a planar microscope slide, fixed to prevent cell loss or degradation, and stained in a manner that accentuates and differentiates the various cellular structures. These prepared samples are subjected to detailed microscopic evaluation by a cytotechnologist or pathologist to detect and classify any cellular abnormalities that may be present in the cells deposited on the microscope slide. The results of these evaluations are reported to the attending physician who determines whether additional evaluation or treatment of the patient is required.

The Pap test as it is currently practiced is time consuming and requires a highly skilled supporting infrastructure. Even in countries with the necessary infrastructure, several weeks can elapse between the taking of the sampling and the reporting of the results of the evaluation to the attending physician. The uncertainty attendant in this delay is stressful to the patient. As it is not practical for the patient to be retained at the medical facility until the results of the evaluation have been returned, it is necessary for the attending physician to contact the patient to inform them that the results of the test were negative or, conversely, if the results were positive, to arrange for a follow-up visit.

In the US, fewer than 60% of the patients contacted with positive results actually present themselves for follow-up evaluations or treatment. This percentage is lower in other countries and is particularly low in public health screening programs and clinics that deal predominantly with transient populations and populations that are remote from the site of testing. Furthermore, depending upon the particular patient population, between 50 and 90 percent of all Pap samples taken are determined to contain no evidence of cellular abnormalities. This high percentage of negative samples imposes a substantial burden on the health care system and diverts resources from making cervical screening tests more widely available.

It is therefore desirable to provide a means of cervical screening that can produce a determination of whether a sample does or does not contain evidence of cellular abnormalities within the time frame of a typical cervical examination. As such a means provides the test results before the patient leaves the examination area, the uncertainty and stress of waiting for a negative diagnosis is eliminated and patients showing positive results can be retained for immediate follow-up and treatment.

Identifying those patients showing no signs of cellular abnormalities at the time of the initial examination also reduces the number of samples that must be sent to a laboratory for evaluation. This reduces the non-productive burden on the health care system and frees resources that can be used to increase the availability of cervical screening and other diagnostic testing.

The manner in which a positive result is followed up varies substantially by country. In some countries such as the US, a finding of ASCUS or higher is generally considered to be grounds for follow-up or medical intervention. In other countries, the standard of care is to follow up or intervene in cases where the detected degree of abnormality corresponds to LSIL (or HSIL) and higher, but, in recognition that many lower grade abnormalities are benign or revert to normal over time, to ignore lower grade detected abnormalities. It is therefore desirable to be able to establish a reporting threshold that is consistent with the prevailing standard of care.

Cervical abnormalities generally present in the form of lesions or localized clusters of abnormal cells. The sampling methods utilized in current cervical screening procedures acquire cells from these lesions, but then disperse these cells into a typically much larger number of normal cells obtained from outside of the boundaries of the lesion. This dispersion results in the evaluation of a conventional cervical sample being an exercise in the detection of a rare event, that is, finding one or a few abnormal cells within a background consisting of a very large number (50,000-300,000) of normal cells. Dispersion also precludes using the sample to determine the location of the lesion on the cervix.

It is therefore desirable that a means of sampling and evaluation be provided that retains the spatial relationships that exist between the cells in-vivo. Retaining these relationships effectively eliminates dispersion and allows mapping of the test results onto the cervix for the purpose of guiding follow-up or intervention.

### SUMMARY OF THE INVENTION

Accordingly, the present invention is directed to evaluating a cellular sample in a manner in which the spatial relationships existing between said cells in-vivo are preserved and can be used to guide subsequent follow-up and treatment where abnormalities are detected. The present invention is directed to providing a means whereby the decision threshold at which a sample is declared to be abnormal and medical follow-up is warranted can be adjusted in accordance with the prevailing standards of medical care.

Therefore, the invention is found in an instrument for analyzing cervical cells. The instrument includes receiving means for receiving a topological array of cervical cells, reagent application means for applying one or more reagents to the topological array of cervical cells, analysis means for optically analyzing the topological array of cervical cells, and reporting means for reporting results of the analysis means.

The invention is also found in a method of analyzing cervical cells. The method includes steps of collecting the cervical cells on a non-planar surface, wherein the cervical cells are arranged on the non-planar surface in accordance with their orientation prior to collection, staining the cervical cells with an appropriate reagent while the cervical cells remain on the non-planar surface, and analyzing the cervical cells while the cervical cells remain on the non-planar surface.

Other features and advantages of the present invention will be apparent from the following detailed description and drawings.

### BRIEF DESCRIPTION OF THE DRAWINGS

FIG. 1 is a sectional view of a cell collection device used in accordance with a preferred embodiment of the present invention.

FIG. 2 is a plan view of the present invention.

FIG. 3 is a sectional view of the sample processing station taken substantially in the plane of line 3-3 in FIG. 2.

FIG. 4 is a sectional view of the reading station taken substantially in the plane of line 4-4 in FIG. 4.

FIG. 5 is a sectional view of the optical system taken substantially in the plane of line 5-5 in FIG. 2.

FIG. 6 is a sectional view of the cell collection device according to FIG. 1, illustrating an optional washing system.

### DETAILED DESCRIPTION OF THE PREFERRED EMBODIMENTS

The present invention is a device for the processing, analysis and classification of cellular materials that are presented to the device on a supporting surface having a non-planar surface topography.

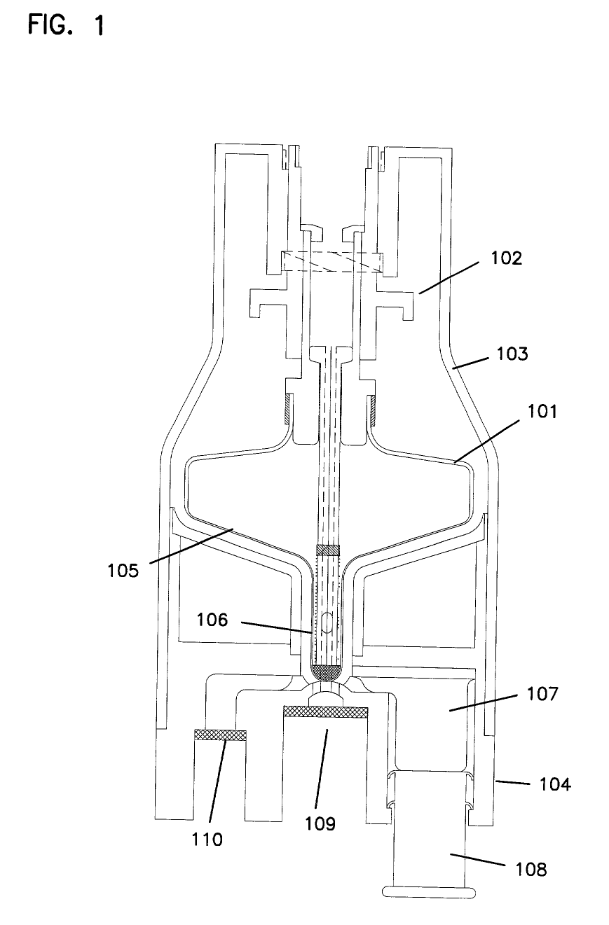

One embodiment of the present invention is intended for use with the cervical cell collection device described in provisional application Ser. No. 60/167,831 and incorporated herein by reference. As seen in FIG. 1, the cell collection device 100 includes an elastomeric sample collection element 101 having a topography that preferably conforms to the shape of the human cervix; a device 102 for coupling the sampling element 101 to an external mechanism; a multifunctional container 103; and an end closure 104, one face of which, in certain embodiments, may have a topography that approximates a mirror image of that of the sampling element 101.

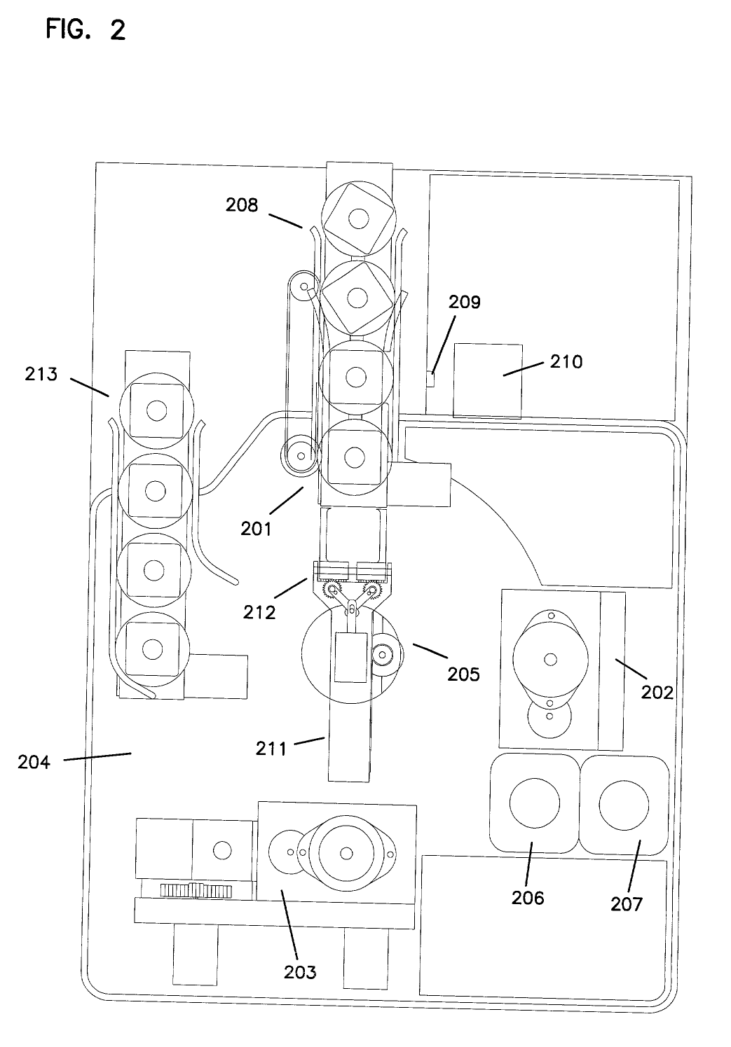

The present invention is illustrated schematically in FIG. 2. The particular embodiment described below implements a cell staining protocol that is based upon the use of a primary antibody to which a fluorophore has been conjugated. The cellular sample is delivered to the instrument contained within the sample collection device 100. A cell fixative solution within the sample collection device preserves the cells and preconditions them for staining. Upon transfer from the loading station 201 to the processing station 202 of the instrument, the fixative can be drained from the cell collection device 100 into a waste container 207 and the residual fixative is removed by exposing the cells on the supporting surface of the cell collection device 100 to a wash solution that is provided from an external bulk container 206.

The immunohistochemical fluorescent staining reagent is then applied to the cells from a compartment 107 located within the closure element 104 of the sample collection device 100. After the staining reaction is completed, the used staining reagent can be drained from the reaction chamber, the adhered cells can be washed as before to remove residual staining reagent, and the stained cells can be dried on the surface of the cell collection element 101. The cell collection element 101 with adhering stained cells can then be transferred to a reading station 203 where the locations and fluorescent intensities of any fluorescent objects on the surface of the cell collection element 101 are determined in a spatially resolved manner. The acquired spatially resolved fluorescence data can be analyzed and the results of the analysis reported in graphical and/or numerical format as specified by the user. Processed cellular sampling devices can be delivered to the user for disposal or secondary processing.

One skilled in the art can readily discern means to extend, enhance and adapt the embodiment described. For example, although the embodiment described utilizes both unit dose and bulk reagent delivery methods, all reagents could be delivered in either unit dose or bulk form depending upon the needs of the present application. Similarly, although a single step fluorescent immunohistochemical staining protocol using a labeled primary antibody is described, protocols utilizing primary, secondary and higher level antibodies, avidin-biotin binding technology, DNA probes, enzymatic signal amplification and similar staining procedures can be implemented. The present invention is also not limited solely to fluorescent immunohistochemical staining procedures. Chromatic histochemical staining procedures can, for example, also be employed. Alternatively, the intrinsic auto fluorescence of cellular materials may be used for the classification of these samples. These and other enhancements, extensions and adaptations, either individually or in combination, lie within the spirit and scope of the present invention.

As shown in FIG. 2, samples are introduced into the instrument via a loading station 201. The loading station 201 provides a point where multiple samples can be queued for entry into the instrument and serves as a barrier that isolates the processing section of the instrument from external influences. Other functions can also be performed by the loading station to prepare the sample for processing, and to provide a measure of protection against certain types of operational faults and errors.

The queuing function of the loading station serves two primary purposes. One of these purposes is that it decouples operator intervention from the timing cycle of the sample processing. In the absence of a queue, the operator may only introduce a new sample into the system at those times during the processing of the previous sample when a position at the processing station 202 is available. A queue, however, allows new samples to be introduced at any time subject only to the limitation of queue capacity.

The second purpose of the queuing function is to allow for the preconditioning of the samples before they enter the processing section of the instrument. Environmental parameters such as the temperature at which the staining reaction is performed have a substantial effect upon the time required to perform the staining, the degree of staining obtained and, in some cases, the ability of the stain to discriminate between various cellular constituents. To ensure consistency in the processing of samples, and thus to ensure that the results obtained from the processed samples can be compared on a common basis, those environmental parameters that affect the processing results must be controlled within suitable limits.

For example, the temperature within the processing section 204 of the instrument is controlled within the range of 35±0.5° C. even though the operating temperature range outside of the processing section may vary between 15° C. and 30° C. Introducing samples, reagents and other materials at the external ambient temperature into processing section 204 causes thermal transients within the processing section that should be eliminated or minimized before processing is initiated. Preconditioning the samples and reagents at a temperature that approximates the desired operating point minimizes the magnitude of the transient and, therefore, the time in processing section 204 required to bring the temperature to within the desired limits. To this end, the loading station 101 incorporates a standard heating element and blower (not shown) that bathes the samples waiting in the queue with warmed air at a temperature that is approximately equal to the desired operating point temperature. When these pre-warmed samples enter the processing area, only minor temperature adjustments are required to bring them to the desired operating point.

Similarly, the loading station 201 serves to isolate or buffer the processing section 204 from the external environment because the processing section can be completely enclosed except for a small port through which samples are transferred. A door, hatch or load lock mechanism of known design can be used to close the transfer port when samples are not being transferred should this added degree of isolation be preferred in a particular application. The loading station 201 eliminates the need for a user to directly access the processing section 204 and therefore eliminates or reduces the environmental transients associated with such direct access.

The loading station 201 also works to minimize the potential for operator error. Preventing direct operator access to the processing section 204, for example, eliminates the possibility that the operator may interrupt the process flow at in inappropriate time or cause other disruptions to the process. The loading station 201 also incorporates geometrical and other features 208 that ensure that the sample has been properly oriented and configured for introduction into the system.

By way of example, one version of the cell collection device 100 described in provisional application Ser. No. 60/167,831 incorporates a plunger mechanism that must be manually depressed to dispense a fixative solution onto the collected sample. Failure to completely depress the plunger may result in incomplete or no fixation of the sample with consequent failure of the staining process. The loading station is arranged with known mechanisms so that if this plunger has not been completely depressed prior to the attempt to load the sample into the loading zone, the projecting plunger prevents the sample container from entering the loading station and thus rejects the sample until the plunger has been fully depressed. Similar gates ensure that the collection device handle has been detached; that the collection device has been properly closed; and that it has been introduced to the loading station in the proper orientation.

As was previously described, the fixative solution must remain in contact with the collected cells for at least a certain minimum period of time in order to ensure that the collected cells will be properly stained. This minimum fixation period depends upon multiple factors including, for example, the type of staining to be performed. By way of example, more extensive cell fixation and permeabilization are required for a sample being treated with a stain directed against a constituent of the cell nucleus than for a sample where the stain is being directed against an extracellular membrane constituent. Fixation may, in the latter case, be completed in a matter of seconds while, in the former case, adequate fixation and permeabilization may require tens of minutes. Certain versions of the cell collection device described in provisional application Ser. No. 60/167,831 incorporate a liquid-based timing mechanism (not shown) that undergoes a change in color and reflectivity when the sample has been exposed to the fixative for an adequate period of time. Sample transfer from the loading station 201 to the processing section 204 is conditional upon detection of this change by a reflectance sensor 209 incorporated into the loading station 201, thus ensuring that only adequately fixed samples are presented to the processing station 202 for staining. Samples that do not exhibit this reflectance change within a specified period of time after entering the loading station are rejected by the system.

The sample container 103 can incorporate known labeling (not shown) that can communicate certain information. This labeling can, by way of example, carry information in machine readable form such as a bar code concerning the patient identification as well as the type of test to be performed, the lot and serial numbers of the sampling device, and the expiration date of the sampling device. The loading station 201 can incorporate a suitable means 210, such as a bar code scan engine or an imaging device, for reading this information from the label.

Such information is used in multiple ways by the present invention. The patient identification and sampling device serial number, for example, provide a means of linking a particular patient to a particular sample, and to the report that results from the processing and analysis of that sample. Information pertaining to the type of test allows the system to adjust various operating parameters such as reaction times and reagent volumes to suit the test to be performed while the sampling device lot number can convey calibration information that is specific to the collection device and reagents that are being used. The expiration date permits the system to reject samples where the collection device has exceeded its expiration date and is therefore suspect.

A transfer mechanism 205 of known design is employed to move the sample between stations in the instrument. In the embodiment being described, the same transfer mechanism is employed to move the sample between the various stations. If desired, multiple transfer mechanisms can be employed. However, the functions of the processing and reading stations can be integrated into a single station, thus obviating the need for a means of transferring a sample between them. The preferred embodiment for a specific instance of the invention depends upon the particular application environment in which the invention is to be used.

The illustrated embodiment, for example, is intended for use in an environment where relatively few samples are to be processed and throughput, as measured in samples processed and read per unit time, is not a major consideration. Such an environment can be found in the practice of a solo physician or small group of physicians. Conversely, a large group practice, a reference laboratory, or a public health screening program typically requires a high throughput. An embodiment of the present invention intended for use in such a high throughput environment will typically incorporate multiple processing stations and multiple transfer mechanisms. In the highest throughput environments, there may be multiple instances of each of the station types within a single instrument. Each configuration imposes different requirements and thus design constraints on the transfer mechanism(s).

In the present embodiment, there is a single instance of each station type and the corresponding transfer mechanism is as shown in FIG. 2. In this instance, the transfer mechanism 205 takes the form of a single arm 211 having an end effector 212 that is capable of grasping and releasing the sample 100, and which is capable of radial, rotational and altitudinal motions. In operation, the arm 211 rotates about a central vertical axis until the arm 211 is aligned with the sample 100 to be transferred from the initial station. The arm 211 then extends radially until the end effector 212 contacts and grasps the sample 100. Raising the arm vertically lifts the sample until it is no longer in contact with the station being addressed and the sample can be withdrawn from the station by retracting the arm 211 in the radial direction. The sample 100 is moved to the target station by rotating the arm 211 about its central axis until the sample 100 is aligned with the target station; extending the arm 211 radially until the sample 100 is properly positioned within the target station; lowering arm until the sample 100 is in contact with the target station; releasing the sample 100 from the end effector 212; and retracting the arm 211 in the radial direction.

The processing station 202 in the present embodiment is intended for the processing of sample collection devices such as are described in provisional application Ser. No. 60/167,831. Other sample collection devices may require corresponding changes to the design and operation of the processing station. As previously noted, it is assumed that the sample is immersed in fixative solution when it is delivered to the processing station and it is further assumed that the single staining reagent is located in a compartment within the sample collection device and that the wash solution is provided from a bulk supply associated with the instrument.

A suitable processing station for this configuration is illustrated in FIGS. 1,2 and 3. The sample 100 is delivered to the processing station 202 completely encased in a container that must be opened before the sample can be processed. The transfer mechanism 205 delivers the sample collection device to the processing station 202 in an orientation such that three access ports 108, 109 and 110 in the end closure 104 and the coupling element 102 on the opposite face of the sample collection device are aligned with the corresponding features in the processing station. Additional features in the processing station mate with ridges and flats (not shown) incorporated into the outer surface of the sample collection device 100 in a manner that allows clamp mechanisms 303 and 304 to secure the container 103 and end closure 104, respectively, to the processing station 202.

When the collection device 100 is properly positioned and is secured in the processing station 202 by clamp 304, a shaft 301 is extended from the processing station 202 and engages the coupling element 102 on the collection device. Rotating shaft 301 disengages the sampling element 101 from the shell 103 of the collection device 100 and separates the shell 103 from the end closure 104. Slightly retracting shaft 301 lifts the shell 103 from the end closure 104 to a position where the shell 103 is secured in place by clamping mechanism 303. This leaves the sampling element 101 suspended above the end closure 104, the face 105 of which is contoured in a manner that mirrors the contour of the face of the sampling element 101. The contoured face of the end closure 104 forms a well into which reagents can be introduced and into which the sampling element can be dipped for the purpose of performing the staining and washing reactions.

At this point, the reaction well contains the fixative solution that was applied to the sample prior to introduction of the sample into the instrument. As illustrated in FIG. 1, this fixative is drained from the well through the central port 109 in the end closure. The well is then filled with wash solution from the bulk supply via a second port 110 in the end closure 104 and the sampling element 101 lowered until the face of the element is immersed in the wash solution. Washing can be facilitated by rotating or oscillating the sampling element longitudinally or, alternatively, by introducing a pulsating stream of air into the sampling element via a channel in the coupling element. Upon completion of the washing, the sampling element is raised above the well and the used wash solution is drained as before. The wash cycle may be repeated as needed until the excess fixative has been removed from the sampling element.

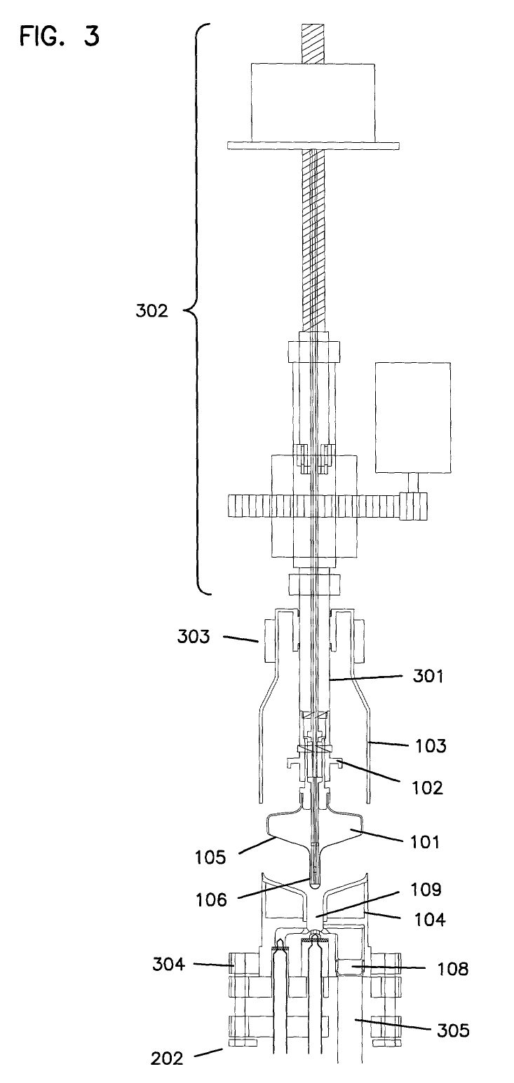

Upon completion of the removal of the fixative solution, the staining reagent is introduced into the reaction well. In the configuration illustrated, the reagent is contained in a break-seal pouch 107 within the end closure 104 of the sampling device 100. As suggested in FIG. 3, the instrument extends a plunger 305 through an access port 108 in the end closure 104 of the sampling device 100 to compress the reagent pouch 107. This pressure causes the break-seal to rupture, thus discharging the contents of the pouch into the reaction well. The staining reaction is carried out by immersing the sampling element into the pool of reagent in the reaction well and mixing as described above. At the conclusion of the staining reaction, the sampling element is elevated above the reaction chamber, the spent reagent is drained from the chamber, and the sampling element washed as described above. The washed sampling element can be dried in a stream of warm, dry air. Other dispensing means such as spraying, aspirating, nebulizing or pipetting may be employed to deliver fixative, staining reagents and wash solution to the sampling element 101. Such alternative dispensing means will require that the design and operation of the process station 202 will differ from that described above.

The preceding description assumes that the sampling element 101 is positioned directly above the reaction chamber such that the entire face of the sampling element 101 can be simultaneously immersed in the reagent pool. This arrangement is acceptable for many applications. However, in some applications the reagent(s) are expensive, in short supply, or, for other reasons, must be conserved. The volume of reagent required to process a sample can be reduced by tilting the sampling element 101 away from the vertical and making corresponding changes in the shape of the face of the end closure (reaction well). In this configuration the well is filled only to a level that ensures at least a continuous line of contact between the reagent and the sampling element. Rotating the sampling element about its axis of symmetry causes the reagent to coat the entire surface of the sampling element. The optimum tilt angle to minimize the consumption of reagent is determined by the shape and dimensions of the sampling element. In the case of one particular type of sampling element, it was possible to reduce the volume of reagent required by approximately 60% by tilting the sampling element by 50 degrees away from the vertical.

The rate of drying of the sampling element is determined by the amount of residual fluid on the surface of the sampling element and by the volatility of this fluid. The drying rate can be increased by rinsing the sampling element in a water miscible, volatile cell fixative solution such as ethanol or isopropanol prior to drying.

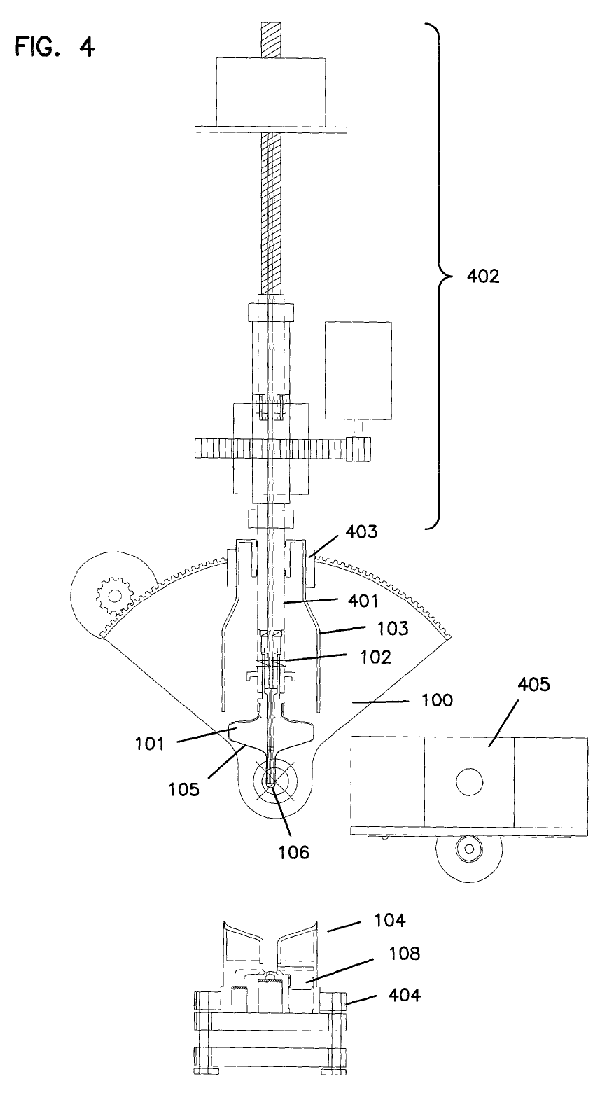

Upon completion of the staining, washing and drying, the sample container is re-closed, the sampling device 100 is disconnected from the vertical shaft 301, and the device 100 is moved to the reading station 203 by the transfer mechanism 205. The reading station 203 is illustrated more fully in FIG. 4. When the collection device 100 is properly positioned and secured, by clamp 404, in the reading station 203 (FIG. 2), a shaft 401 extends from the reading station 203 and engages the coupling element 102 on the collection device 100. A rotating shaft 401 disengages the sampling element 101 from the shell 103 of the collection device 100 and separates the shell 103 from the end closure 104. The retracting shaft 401 lifts the shell 103 from the end closure 104 to a position where the shell 103 is secured in place by a clamping mechanism 403. This suspends the sampling element 101 a sufficient distance above the end closure 104 to provide the reader optics 405 with the necessary access to the stained cellular material on the surfaces 105 and 106 of the sampling element 101.

The primary function of the reading station 203 is to measure the fluorescence at each point on the surface of the face of the sampling element 101. To this end, either of two optical systems are used depending upon whether, in the particular application, it is necessary or desirable for the operator to be able to visually view the surface of the sampling element. If, as is the case in research and certain other specialized applications, it is desirable for the operator to be able to visually view the surface of the sampling element, the optical system consists of a video camera or viewing tube coupled to an appropriate microscope objective lens.

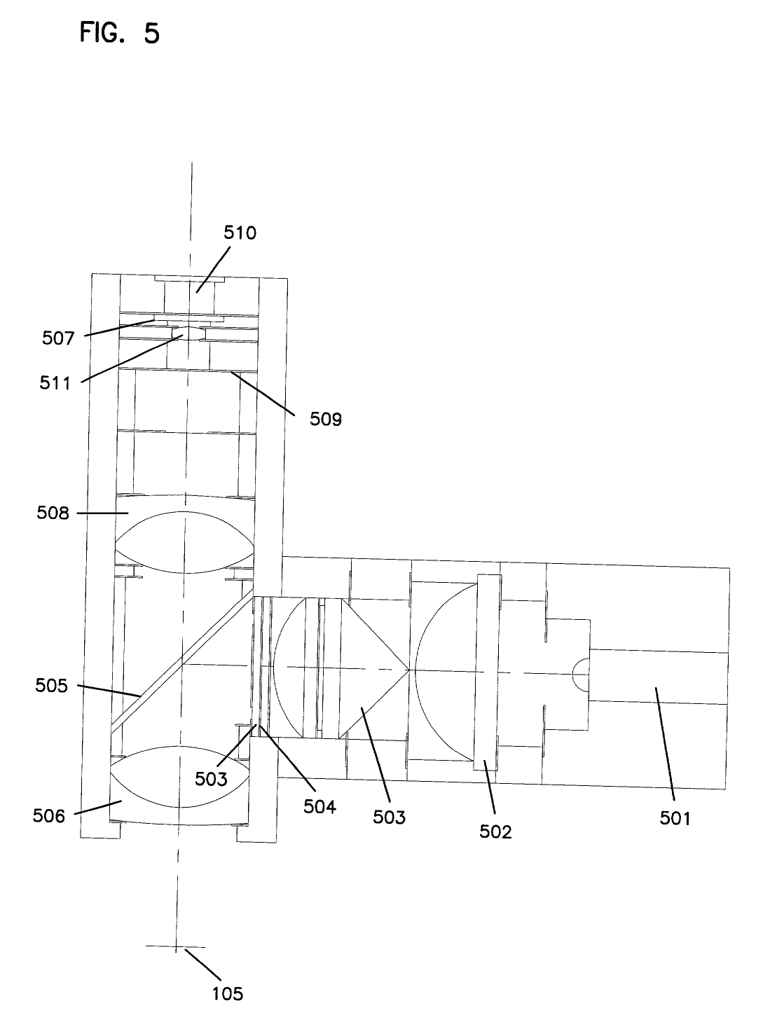

In applications where it is neither necessary nor desirable for the operator to view the surface, a flying spot scanning optical system is employed. The flying spot optical system shown in FIG. 5 is of a dark field, epi-illumination confocal design having approximately ten micron spatial resolution at the surface of the sampling element. Proper selection of the light source 501 and wavelength selection filters 504 and 507 and dichroic mirror 505 allows this optical system to be used with fluorescent immunohistochemical staining reagents incorporating any desired fluorophore. Other selections allow the use of nonfluorescent reagents such as standard histochemical stains.

In this design, light emitted from light source 501 is collimated by a lens 502 and converted to a collimated ring of light by an axicon 504. Interference filters 503 are used to select the excitation wavelength. A dichroic mirror 505 redirects the illumination such that it is coaxial with the optical axis of the scanning optics. A high numeric aperture doublet lens 506 used as an objective focuses the illumination on the surface 105 of the sampling member 101. The illumination incident on objective lens 506 is in the form of a ring that passes through the periphery, but not the center of the lens. The relationship between the inner and outer diameters of this ring of light and the numerical aperture of the objective lens 506 are selected to establish dark field illumination conditions where specular reflections from the surface of the sampling element do not reenter the central portion of the objective lens. This increases the signal to noise ratio in the detected signal and relaxes the performance requirements placed on the detection optics.

Fluorescent light emitted by the sample is collected and approximately collimated by the central portion of the objective lens 506. This collected collimated light passes through the dichroic mirror 505 and is focused by a second doublet lens 508 that is identical to the objective lens 506 upon pinhole aperture 509 which defines the size of the sampling spot on the surface of the sampling element 101. Light passing through aperture 509 is collimated by collimating lens 511 and the fluorescence emission wavelength to be detected is selected by interference filter 507. Photodetector 510 converts the incident light energy into an electrical signal. In some applications, it is desirable to replace filter 507 with an optical subsystem that allows the simultaneous detection of emitted light at multiple wavelengths.

The shape, reproducibility and stability of the sampling element 101 and the type of optical system determine the characteristics required of the positioning system that is employed in the reading station 203. The function of the positioning system is to move sampling element 101 in such a manner that the optical axis of the reader optics 405 traces a prescribed path over the entire face 105 and tip 106 surface of sampling element 101. In order to minimize measurement errors, the optical axis must be perpendicular to the surface of the sampling element 101 at each point on said surface. Furthermore, the surface of the sampling element 101 must remain within the depth of focus of the optical system throughout the scanning process. The resolution, accuracy and precision required of the positioning system is determined by the type of optical system employed. An optical system based upon a CCD video camera, for example, imposes less stringent demands upon accuracy, precision and resolution than does a flying spot scanner because, in the former case, the effects of several of the predominant types of positioning error can be compensated for during the processing of the acquired data. Similarly, the means (not shown) employed for determining the position and alignment of the optical system 405 relative to the surface of sampling element 101 depends upon the type of optical system employed. The embodiment illustrated in FIG. 4 utilizes two linear and two rotary axes of motion, all under servo control, to accomplish these ends. An alternative optical system that does not provide spatially resolved data, and thus does not require precise positioning of sampling element 101 relative to the reader optics 405, can also be envisioned.

The electrical signal produced by the photo detector 510 is amplified, filtered and digitized by means not shown to produce a numerical representation of the distribution of fluorescence on the surface of the sampling element. This numerical data is processed to detect the presence of abnormal cells on the surface of the sampling element 101 and to classify such abnormal cells as may be detected.

Known calibration standards and procedural controls may be incorporated into the system to ensure that the optical system and detection electronics have been properly adjusted and that the staining reaction has been performed successfully. A typical calibrator may consist of a material such as fluorescent microparticles having known characteristics that are disposed in such a manner, including but not limited to locations on the surface of the sampling element, that the material can be viewed and quantitated by the optical system. A typical procedural control may, by way of example, consist of a material that absorbs or reacts with the staining reagent in such a manner to give an optical signal of a predetermined level if the sample staining reaction has been properly performed.

After background correction and normalization, a histogram of the values of the collected data points is constructed and analyzed to determine the threshold data value that discriminates between normal and abnormal cells. This threshold value is constrained to ensure that the gradient search method used to locate the threshold value has converged on an acceptable value that is consistent with the values determined during the characterization of the staining reagent. The threshold is used to select those data points having signal levels that may indicate the presence of an abnormal cell.

The thresholded data is then processed to aggregate groups of data points that are in proximity to one another into “objects”. The area of each object, defined as the number of data points included within the boundaries of the object, the average of the values of these data points, the ratio of the average data value to the area of the object, and other parameters are computed. As the optical system is designed such that each collected data element is smaller than the smallest abnormal cell of interest, the area of any detected object that may be a cell must be greater than a particular value that is determined during system calibration. Any object having an area that is smaller than this predetermined value is rejected as being non-cellular. Other morphological parameters such as the length to width ratio of an object may also be used in the discrimination between cellular and non-cellular materials. Characterization of the staining reagent establishes the range of average data values corresponding to abnormal cells.

Objects having average data values outside of this range are rejected as being non-cellular. The remaining objects are presumed to be abnormal cells that may, if desired, be further classified on the basis of object area, average data value and other parameters so as to indicate the degree of abnormality of the cell.

Some fluorescent immunohistochemical reagents do not, by themselves, provide sufficient discrimination between normal and abnormal cells, between cell types or between cellular and non cellular materials to meet accepted clinical standards. In these cases, multiple reagents each having different specificities and distinguishable fluorescent properties are applied to the sample and quantitated by the optical system. The reagents in such a combination may, for example, be directed against different antigenic determinants in the cells or an immunological reagent directed against a particular determinant may be combined with a reagent such as propidium iodide that discriminates between cellular and non-cellular materials. The data analysis process is extended to select and classify objects based upon measurements made of these combinations of fluorophores.

The results of the data analysis are reported to the clinician in a form that reflects the needs of the particular application. Samples taken as part of a screening program may, for example, be reported as being within normal limits if no abnormal cells are detected, or marked to indicate the presence of abnormal cells. In a diagnostic application, abnormal cells may be reported in a manner that reflects the degree of abnormality detected. One typical report is a printout or visual display that lists the findings of the analysis and other desired information such as patient identification. The report may include the classification of each abnormal cell detected or it may provide a classification for the entire specimen based upon a composite of the classifications of the individual abnormal cells detected.

The sample collection and analysis process described above preserves the spatial relationships that existed between the cells before collection. In other words, cells collected from a lesion (a group of abnormal cells) will be clustered together on the surface of the sampling element. As the sampling, staining, and data analysis processes retain this spatial information and this spatial information can be linked to an absolute physical location on the sampling element in a manner that allows this location to be linked to a specific position on the cervix, the analytical results can be presented in the form of a map that displays the location of the detected abnormality on the cervix. The clinician can use this map to guide whatever confirmatory and follow-up procedures are indicated to the specific location(s) on the cervix where the abnormalities exist.

The cells processed using the present invention can be released from the surface of the sampling element and deposited on the surface of a conventional microscope slide using known liquid-based slide preparation techniques. These slides may then be used for the purposes of confirmation of results, additional testing or cytological diagnosis using other methods. The fluorescent immunohistochemical stains applied to the cells by the present invention are retained by the cells upon transfer to a microscope slide. Under certain conditions—specifically that subsequent staining processes do not suppress or mask the fluorescence of these stains—these fluorescently stained cells can be visualized by fluorescence microscopy. This fluorescence can guide the cytologist to specific regions of the slide where abnormal cells are present and may thus facilitate evaluation of the slide.

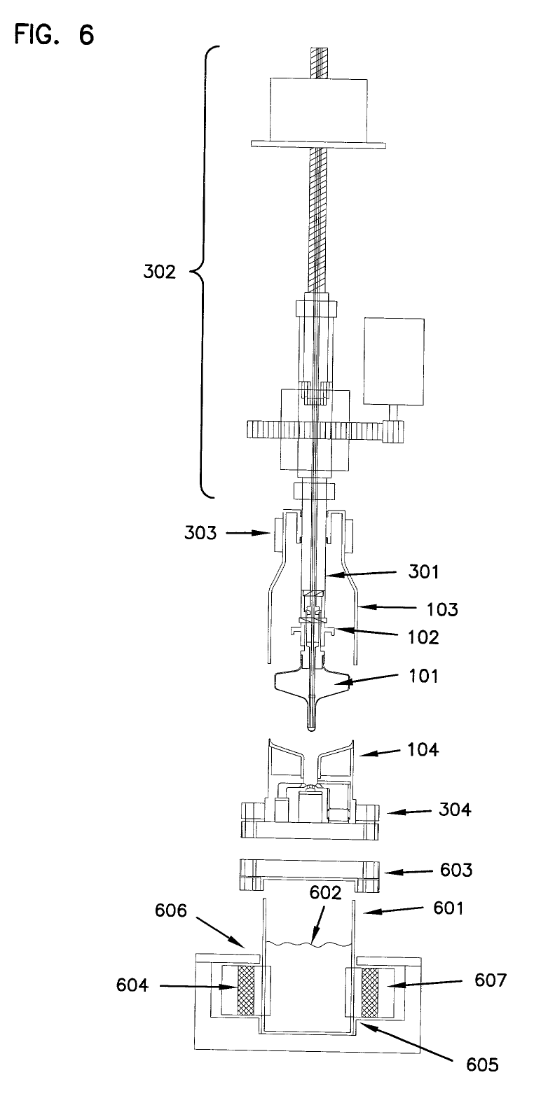

In a preferred embodiment, the cells are removed using an apparatus as illustrated in FIG. 6. A user inserts a capped vial of preservative solution into a well 606 in the base of the instrument and then inserts a sealed canister containing cells into a slot (not shown) in the housing face of the instrument that is located directly above the well for the preservative solution. A vial clamp 607 secures the vial in the well.

A rotary decapping mechanism 603 extends from the instrument over the vial; is lowered to make contact with the vial cap; grasps and unscrews the cap; raises the cap above the vial; and retracts the cap into the instrument. A plug removal mechanism 304, which, in its extended configuration, state forms the bottom of the slot into which the canister is inserted, clamps the flange of the plug securing it to the instrument. A clamp 303 secures the shell of the canister drive mechanism 302, and extends shaft 301 to engage the coupling 102. This action couples the sampling balloon to the shaft and disengages the balloon/coupling from the canister shell.

The drive mechanism 302 then elevates the canister and balloon until it clears the plug 104, which is then retracted into the instrument. The drive mechanism 302 lowers the balloon 101 into the preservative solution 602.

Mechanical energy is applied to release cells from the surface of the balloon 101 into the preservative solution 602. This energy may be applied by ultrasonic excitation applied to the preservative solution. In this mode, ultrasonic transducers 604 are embedded in the clamp mechanism 607 and ultrasonic energy is coupled through the walls of the vial 601 into the solution 602 which, in turn, couples that energy to the surface of the balloon 101. Alternatively, linear and rotary oscillation can be imparted to the balloon 101 by the drive mechanism 302. In yet another embodiment of the invention, pneumatic excitation can be applied to the interior of the balloon 101 via a path through the drive mechanism 302 and the coupling 102. The pneumatic excitation is provided in the form of pulsating air pressure (nominally less than 5 psi per pulse). The pulsating air pressure may be provided by any appropriate means known to those skilled in the art including, but not limited to reciprocating piston, peristaltic and astable air amplifier devices.

The drive mechanism 302 then raises the balloon 101 to above the level of the plug 104. The plug 104 is extended from the instrument and the clamp mechanism 303 is released. The drive mechanism 302 lowers the balloon 101 and the canister shell 103 onto the plug 104. The drive mechanism 302 disengages the shaft 301 from the coupling 102 and simultaneously engages coupling 102 to the canister shell 103. The drive mechanism 302 retracts shaft 301 leaving the assembled canister in the slot on the instrument face.

The clamp 304 then releases, thus allowing the user to remove the canister with enclosed balloon from the instrument. The rotary decapper 603 extends from the instrument; lowers the cap onto the vial 601; secures the cap to the vial; elevates to clear the vial and retracts into the instrument. The clamp 607 releases, thus allowing the user to remove the sealed vial 601 and enclosed cell suspension from the instrument. The vial containing the cell suspension may then be processed by any relevant method known to those skilled in the art.

While the invention has been described with reference to specific embodiments, it will be apparent to those skilled in the art that many alternatives, modifications and variations may be made. Accordingly, the present invention is intended to embrace all such alternatives, modifications and variations that may fall within the spirit and scope of the appended claims.

We claim:

1. An instrument for analyzing cervical cells, comprising:
receiving means for receiving a topological array of cervical cells;
reagent application means for applying one or more reagents to the topological array of cervical cells;
analysis means for optically analyzing the topological array of cervical cells; and
reporting means for reporting results of the analysis means.

2. The instrument of claim 1, wherein the topological array of cervical cells is arranged on a non-planar surface.

3. The instrument of claim 2, wherein the topological array of cervical cells is spatially arranged in accordance with a topological arrangement of the cervical cells before they were provided on the non-planar surface.

4. The instrument of claim 1, wherein the cervical cells comprise cervical epithelial cells.

5. The instrument of claim 2, wherein the reagent application means apply a suitable reagent to the topological array of cervical cells while the cells are arranged on the non-planar surface.

6. The instrument of claim 2, wherein the reagent application means apply a reagent that imparts fluorescence to the array of cervical cells in such a way as to differentiate normal cervical cells from abnormal cervical cells.

7. The instrument of claim 6, wherein the reagent that imparts fluorescence to the array of cervical cells indicates a degree of abnormality of the cervical cells.

8. The instrument of claim 5, wherein the reagent application means apply the suitable reagent to the topological array of cervical cells by dipping the non-planar surface into a pool comprising the reagent.

9. The instrument of claim 5, wherein the reagent application means apply the suitable reagent to the topological array of cervical cells by rotating the non-planar surface through a pool comprising the reagent.

10. The instrument of claim 5, wherein the reagent application means apply the suitable reagent to the topological array of cervical cells by pulsating the non-planar surface in a pool of reagents comprising the reagent.

11. The instrument of claim 5, wherein the reagent application means apply the suitable reagent to the topological array of cervical cells by spraying the reagent onto the non-planar surface.

12. The instrument of claim 5, wherein the reagent application means apply the suitable reagent to the topological array of cervical cells by nebulizing the reagents onto the non-planar surface.

13. The instrument of claim 1, wherein the topological array of cervical cells is contacted with a fixative prior to being contacted with a reagent.

14. The instrument of claim 13, wherein the topological array of cervical cells is contacted with a wash solution after being contacted with the fixative.

15. The instrument of claim 2, wherein the suitable reagent is at least one reagent selected from the group consisting of a histological stain, an immunohistological stain, a DNA probe, a chromogenic stain, a calorimetric stain, and mixtures thereof.

16. The instrument of claim 2, wherein the analysis means comprise an optical analysis device that acquires data describing the cervical cells.

17. The instrument of claim 16, wherein the optical analysis device measures light that is reflected from the non-planar surface.

18. The instrument of claim 16, wherein the optical analysis device measures cellular autofluorescence.

19. The instrument of claim 16, wherein the optical analysis device measures extrinsic fluorescence.

20. The instrument of claim 16, wherein the optical analysis device measures electromagnetic emissions from the entirety of the non-planar surface.

21. The instrument of claim 16, wherein the optical analysis device measures electromagnetic emissions from a representative portion of the non-planar surface.

22. The instrument of claim 16, wherein the analysis device further comprises data analysis means for analyzing the data acquired by the optical analysis device.

23. The instrument of claim 22, wherein the data analysis means analyzes the data based upon signal intensity.

24. The instrument of claim 22, wherein the data analysis means analyzes the data based on normalized intensities.

25. The instrument of claim 22, wherein the data analysis means analyzes the data based on morphological methods.

26. The instrument of claim 22, wherein the data that is analyzed is acquired in multiple spectral wavelength ranges.

27. The instrument of claim 22, wherein the data is analyzed in such a way as to ascertain a position of a particular cell on the non-planar surface.

28. The instrument of claim 22, wherein the data is analyzed in such a way as to provide a map of any and all cells of interest present on the non-planar surface.

29. The instrument of claim 1, further comprising cell recovery means for removing the cervical cells from the non-planar surface and placing the cervical cells in a cell suspension.

30. The instrument of claim 1, further comprising cell recovery means for removing the cervical cells from the non-planar surface and placing the cervical cells, their topological arrangement intact, onto a microscope slide for further analysis.

31. A method of analyzing cervical cells, the method comprising steps of:
collecting the cervical cells on a non-planar surface, wherein the cervical cells are arranged on the non-planar surface in accordance with their orientation prior to collection;
staining the cervical cells with an appropriate reagent while the cervical cells remain on the non-planar surface; and
analyzing the cervical cells while the cervical cells remain on the non-planar surface.

32. The method of claim 31, wherein the cervical cells comprise cervical epithelial cells.

33. The method of claim 31, wherein a reagent is applied that imparts fluorescence to the cervical cells in such a way as to differentiate normal cervical cells from abnormal cervical cells.

34. The method of claim 33, wherein the reagent that imparts fluorescence to the cervical cells indicates a degree of abnormality of the cervical cells.

35. The method of claim 31, wherein the reagent is applied to the cervical cells by dipping the non-planar surface into a pool comprising the reagent.

36. The method of claim 31, wherein the reagent is applied to the cervical cells by rotating the non-planar surface through a pool comprising the reagent.

37. The method of claim 31, wherein the reagent is applied to the cervical cells cervical cells by pulsating the non-planar surface in a pool of reagents comprising the reagent.

38. The method of claim 31, wherein the reagent is applied to the cervical cells by spraying the reagent onto the non-planar surface.

39. The method of claim 31, wherein the reagent is applied to the cervical cells by nebulizing the reagents onto the non-planar surface.

40. The method of claim 31, wherein the cervical cells are contacted with a fixative prior to being contacted with a reagent.

41. The method of claim 40, wherein the cervical cells are contacted with a wash solution after being contacted with the fixative.

42. The method of claim 31, wherein the cervical cells are analyzed by analyzing an electromagnetic emission from the cervical cells.

43. The method of claim 42, wherein the electromagnetic emission comprises cellular autofluorescence.

44. The method of claim 42, wherein the electromagnetic emission comprises extrinsic fluorescence.

45. The method of claim 42, wherein the electromagnetic emission is analyzed based upon signal intensity.

46. The method of claim 42, wherein the electromagnetic emission is analyzed based on normalized intensities.

47. The method of claim 42, wherein the electromagnetic emission that is analyzed is acquired in multiple spectral wavelength ranges.

48. The method of claim 42, wherein the cells are analyzed in such a way as to ascertain a position of a particular cell on the non-planar surface.

49. The method of claim 42, wherein the cells are analyzed in such a way as to provide a map of any and all cells of interest present on the non-planar surface.

50. The method of claim 31, further comprising removing the cervical cells from the non-planar surface and placing the cervical cells in a cell suspension.

51. The method of claim 31, further comprising removing the cervical cells from the non-planar surface and placing the cervical cells, their topological arrangement intact, onto a microscope slide for further analysis.
